(19) United States
(12) Patent Application Publication
(10) Pub. No.: US 2006/0116682 A1
(43) Pub. Date: Jun. 1, 2006

(54) Surgical implant and methods of making and using the same

(76) Inventor: Marc N. Longo, Bellarie, TX (US)

Correspondence Address: PAUL S MADAN;MADAN, MOSSMAN & SRIRAM, PC, 2603 AUGUSTA, SUITE 700, HOUSTON, TX, 77057-1130, US

(21) Appl. No.: 11/289,591

(22) Filed: Nov. 16, 2005

Related U.S. Application Data
(60) Provisional application No. 60/629,141, filed on Nov. 18, 2004.

(51) Int. Cl. A61B 17/80; A61F 2/28

(52) U.S. Cl. 606/69; 623/23.51; 623/17.18; 623/23.55

(57) ABSTRACT

A biocompatible and pliable surgical implant for use in repair and rigid fixation of bone fractures and to compensate for a volume loss is disclosed. The implant includes a biocompatible base member and a volume member having a predetermined volume affixed to the metallic base member. The base is adapted to be secured proximate the fractured bone area.

### CROSS-REFERENCE TO RELATED APPLICATION

This application takes priority from U.S. Provisional Patent Application Ser. No. 60/629,141, filed on Nov. 18, 2004.

### BACKGROUND OF THE INVENTION

1. Field of the Invention

This invention relates to implants and more particularly to an implant device for repairing a fractured bone structure and providing a compensation for a loss of volume.

2. Background

Bone fractures in humans and animals may present fracture-related complications. Among such complications is the situation in which bone fragments resulting from a fracture move apart or are crushed, leaving a fracture unable to heal properly without utilizing permanent implants. Often an implant becomes necessary because otherwise the affected bone areas may not align or join properly. Fractures of human facial bones frequently present such characteristics and can be particularly acute at the eye orbit. The eye's internal bone structure is relatively thin and complex in shape and thus in many situations requires surgical procedures to (a) stabilize the fractured internal orbit bone and (b) insert an implant to compensate for a loss of volume, particularly between the orbit floor and the eyeball. Other bone areas, such as portions of the skull including cheeks and forehead may also require similar surgical procedures.

In one type of a surgical procedure of the eye orbit, a metallic biocompatible and pliable plate that is trimmed to an appropriate size and shape is placed at the orbit floor and then secured to the front skull bone by screws. Such a procedure is utilized when a portion of the eye orbit is fractured. In this manner, the plate itself rests on the orbit floor and provides a relatively firm or stiff base. If compensation for a loss of volume is necessary, a porous synthetic implant of appropriate thickness and shape is implanted to compensate for such a volume loss. Although the porous implants provide adequate volume, they can migrate out of the implanted locations, thereby losing their effectiveness. Such implants exhibit an increased likelihood to migrate if there is insufficient stable bone around the porous implant or if the bone is unstable. In such cases, the implant may migrate anteriorly or down into the sinus, creating a need for further surgery to correct for the implant migration. In addition, a shift or migration of the porous material from its implanted location can cause the eyeball to sag or shift, which may also require a subsequent surgical procedure and/or a new implant to repair such a condition.

It is thus desirable to have a biocompatible implant that will (i) provide a necessary support to the fractured or deformed bone structure, (ii) provide a volume to compensate for any diminished volume, such as due to tissue loss, and (iii) not have a tendency to shift after it has been implanted.

The present invention addresses some of the above-noted problems with currently available implants and provides a implant that provides a structural support for a fractured bone structure and compensation for the loss of volume and methods of making and using such an implant.

### SUMMARY OF THE INVENTION

The present invention provides an implant that includes a base plate or a base member that provides structural support to a fractured bone area and a volume member affixed to the base plate to provide compensation for a loss of volume. The base plate may be made from any biocompatible material that will provide the desired structural support including a metallic material such as titanium. The shape, size and thickness of the base plate is chosen to provide for the desired internal fixation of fractures, as a material for stabilization of the bone and as a bone graft support material. The plate may include perforations and may be coated with biocompatible material to inhibit in-growth of tissue into the perforation. The volume member may be a porous member made from any suitable material, including a substantially non-metallic material, such as polyurethane.

The base member may be relatively thin compared to the volume member. The volume member may be affixed to the base member by any suitable manner, including affixing or bonding these members with a suitable adhesive bonding agent or by fusing them together. The implant is placed on the fractured bone area and the base member is secured to an adjacent bone structure in a suitable manner such as with one or more bone screws or any other appropriate attaching device. The base member of the implant will tend to remain in its implanted place where it has been secured and the volume member remains in its implanted location because it is affixed to the base member.

Examples of the more important features of the invention have been summarized (albeit rather broadly) in order that the detailed description thereof that follows may be better understood and in order that the contributions they represent to the art may be appreciated. There are, of course, additional features of the invention that will be described hereinafter and which will form the subject of the claims appended hereto.

### BRIEF DESCRIPTION OF THE DRAWINGS

For detailed understanding of the present invention, reference should be made to the following detailed description of the preferred embodiment, taken in conjunction with the accompanying drawing wherein like elements have been given like numerals and wherein:

FIG. 1 is a perspective view of an exemplary human skull showing a fracture of the right eye orbit floor and a fracture in the skull that may require an implant to provide structural support for the fracture areas and compensation for a loss of volume;

FIG. 2 is a cross-sectional view of an exemplary implant according to one embodiment of the present invention;

FIG. 3 is a top view of the implant of FIG. 2;

FIG. 4 shows a top view of an alternative embodiment of an implant according to the present invention;

FIG. 5 is a top view of an exemplary embodiment of the base plate of the implant of FIG. 2; and

FIG. 6 is a perspective view of the human skull shown in FIG. 2 with an implant made according an embodiment of the present invention placed on the orbit floor and attached to the facial bone.

### DETAILED DESCRIPTION OF PREFERRED EMBODIMENTS

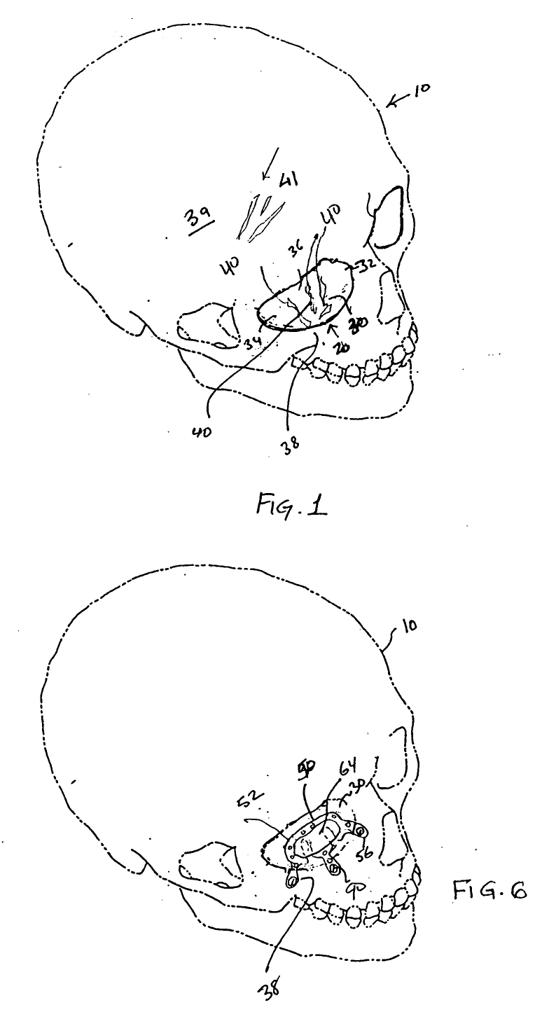

FIG. 1 is a perspective view of a human skull 10 showing the right eye orbit 20 with the orbit floor 30, medial wall 32, lateral wall 34, posterior wall 36, a facial bone structure 38 and the forehead 39. A variety of bone fractures of the eye orbit 20 can occur due to accidents or congenital defects. Such fractures may occur on one or more areas of the orbit floor 30. Bone area 40 on the orbit floor is intended to show only an example of a bone fracture of the orbit floor 30, and represents any fracture type that may require a surgical implant to provide both the structural support to the orbit floor 30 and a certain amount of volume over such a structural support to compensate for a loss of volume for the eye or globe. In reality, many different types of fractures occur in the eye or other areas of the body. Element 41 shows fractures or bone defects in the forehead that may require implants to provide both the bone support and volume compensation. Thus, the apparatus of this invention is intended for use on all such fractures whether on the skull or any other bone area. Also, any volume compensation provided by the implant is desired to remain in the implanted location.

FIG. 2 shows a cross-sectional view of an exemplary implant 50 according to one embodiment of the present invention. The implant 50 includes a base member (plate, strip or panel) 52. The plate 52 may include any number of perforations 54. The plate 52 is usually relatively thin (typically about one mm) and is made from a biocompatible material (i.e., an allopathic material) suitable for use in humans or animals. The plate 52 may be made from titanium or any other suitable biocompatible material. Titanium is an example of widely accepted biocompatible material for such applications. A plate 52 made from titanium, for example, or any other suitable relatively stiff material, can support itself when placed on a fractured area, such as a fractured orbital floor. Platinum is another suitable material and is useful because it has low density and low elastic modulus (stiffness) compared to materials such as stainless steel or cobalt chromium. Titanium plates also are pliable and corrosion resistant. However, for the purpose of this invention any material that will provide the desired or adequate support for the fractured bone portion may be used. Materials such as Teflon, supramid, tantalum, vitallium, polyethylene etc., if suitable, may also be used. Hybrid materials, including metallic and nonmetallic materials, may also be used. A pliable material is desirable because it can be trimmed to a desired shape and size with an instrument such as scissors prior to implanting the implant into the body. The implant also may be made in various anticipated sizes and shapes. The plate 52 may incorporate one or more provisions for securing it to a bone structure such as one or more extensions or fingers 56, having a suitable through-opening or hole 58 for inserting a securing member, such as a bone screw, therethrough. The extension 56 may also be secured to the bone in any other suitable manner.

The plate 52, when placed on the orbit floor 30 and affixed to a bone structure, such as with surgical screws, rests on the orbit floor 30 to provide structural support to the orbit floor. The implant 50 also includes a second member 64 (also referred herein as a volume member) that is attached to a side 65 (usually a top side) of the plate 52. The volume member 64 is attached to the plate 52 in a manner so that the volume member 64 will tend to remain (or will remain substantially) in place (i.e., not shift) relative to the base plate 52 after the implant has been implanted. The combination member also is referred herein as a hybrid implant or device.

The volume member 64 may be attached to the plate 52 by any suitable manner including, but not limited to, by an adhesive 60 or any bonding agent or material or by fusing the volume member on to the plate 52. In another aspect the volume member 64 and the plate 52 may be bonded or attached to each other by a heating mechanism or by an electrochemical reaction. The bonding material may also be of a type that will dissolve over a time period after implantation of the device in the body. As the bonding material dissolves, this allows the body's natural healing properties or mechanisms to ingrow or vaginate and keep the volume member substantially at its implanted position. Examples of such bonding agents include products sold under the trade names “cyanocrylate” glue or “dermabond”.

The volume member 64 may be a porous material having any desired shape and size. In the embodiment shown in FIG. 2, the volume member 64 has a substantially flat bottom surface 63 and a contoured top surface 68 that has sections 64 and 66 of different thicknesses. The volume member's contour and the shape depend upon the amount and dimensions of the volume to be compensated. Typically, the volume member 64 is thicker than the plate 52. The volume member may be a porous member made from a non-metallic biocompatible material such as a polyurethane material. “Medpor,” for example, is such a polyurethane material that is commonly used for compensation of volume in surgical implants. The volume material is usually not compressible by the pressure exerted thereon after the implant. The implant 50, thus, is a hybrid implant that includes a relatively stiff member, usually a metallic member, that provides structural support to the fractured bone and a volume member 64 that provides for the compensation for loss of volume.

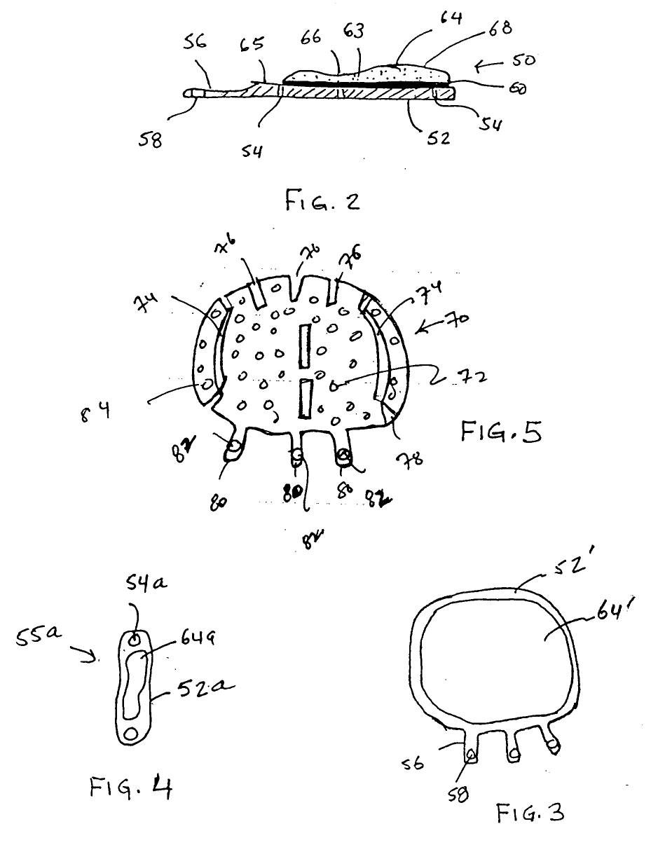

FIG. 3 shows a top or plan view of a hybrid implant that has a base plate 52′ that includes attachment extensions 56 having bone screw holes 58. The volume member 64′ is suitably attached on a surface or side of the plate 52, by man.

FIG. 6 shows another embodiment 55a of a hybrid implant of the present invention. The implant 55a includes a base plate 52a suitable for a small longitudinal fracture having holes 54a for securing it to the bone and a volume member 64a suitably secured to the base plate. The plate 52a has no extensions and may or may not have any perforations therein.

FIG. 5 shows an exemplary embodiment of a base plate 70 that may be used in the present invention. The base plate 70 includes a main section or body that has cuts or openings 74 on each side, opening 76 on the rear side of the plate 70 and opening 78 on the front side. These openings provide flexibility to the plate 70 and allow relatively easy shaping of the plate to match the orbit base or any other fractured bone area. The plate 70 also includes one or more extensions or fingers 80 here shown as an example (on the front side of the plate 70), each such finger having an opening 82 to accommodate a bone screw therethrough. It should be noted that bone screw is one convenient manner to secure the plate to the bone. Any other attachment device or method may be used to secure the plate 52 (FIG. 6) to the bone structure for the purpose of this invention. The plate 52 also may include perforations 72 that permit communication between the bone structure and surrounding tissue mass. As noted above, the plate 70 may be made from pure titanium, which has been determined to be suitable as an implant material or any other suitable biocompatible material. The plate 70 also may be coated with a suitable biocompatible to inhibit the in-growth of tissue in the perforations.

FIG. 4 shows the implant 50 of FIG. 2 placed or implanted in the right orbit of a human skull. A hybrid implant that matches the need for a particular surgery is selected. The selected implant is then shaped, if necessary, and placed on the orbit floor 30 (FIG. 1) or other fractured bone as the case may be. The extensions 50 are then secured to the facial bone 38 (FIG. 1) by bone screws 90 (FIG. 6). Once the plate 52 is secured or affixed to the facial bone 38, the base plate 52 remains in its implanted position. Further, since the volume member 64 (FIG. 2) is affixed on to the plate 52, it will also remain in its initial location without shifting relative to the plate 52. The base plate 52, thus, provides the desired structural support to the fractured bone area and remains in its implanted location because it is secured to the bone structure, and the volume member 64 provides for the loss of volume and remains in its implanted location because it is affixed to the base plate 52.

In general, the hybrid implant may be made in any number of shapes and sizes during manufacturing. Both the volume member and the base plate element may be modified after manufacture to conform to shape and size for individual situations. The volume member of a desired size and shape is affixed to a compliant base plate. The base plate may include one or more provisions for affixing it to a bone structure.

The foregoing description is generally directed to embodiments relating to implants for eye orbit. For the purpose of illustration and explanation the implant of the present invention, however, may be used for any surgical procedure in humans or animals. The base plate may also be of any thickness compared to the volume member. It will also be apparent, however, to one skilled in the art that many modifications and changes to the embodiment set for the above are possible without departing from the scope and the spirit of the invention. It is intended that the following claims be interpreted to embrace all such modifications and changes.

What is claimed:

1. An implant, comprising:
(a) a base member that provides structural support to a bone structure when the base member is placed on the bone structure; and
(b) a volume member affixed to the base member to provide a predetermined volume over the base member.

2. The implant of claim 1 wherein the volume member is affixed on a topside of the base member.

3. The implant of claim 1 wherein the base member is made from a metallic material and has at least one extension that is adapted to be secured to the bone structure.

4. The implant of claim 3 wherein the base member includes a plurality of perforations.

5. The implant of claim 1 wherein the base member is made from titanium.

6. The implant of claim 1 wherein the volume member is relatively porous and is made from one of (i) polyurethane, and (ii) a synthetic material.

7. The implant of claim 1 wherein the volume member is secured to the base member by a biocompatible material.

8. The implant of claim 1 wherein the base member and the volume members are fused together.

9. The implant of claim 1 wherein the base member is pliable and biocompatible.

10. The implant of claim 1 wherein at least a portion of the volume member is greater in thickness than the base member.

11. The implant of claim 1 wherein the base member is made from one of (i) a wire mesh, (ii) pure titanium, (iii) stainless steel, (iv) Teflon, (v) tantalum, (vi) vitallium, (vii) supramid and (viii) a combination of metallic and nonmetallic materials.

12. The implant of claim 1 wherein the volume member is biocompatible.

13. A method of making an implant, comprising:
(a) providing a base member having sufficient strength to provide structural support to a fractured bone structure when placed on an area of the fractured bone structure; and
(b) attaching a volume member on a surface of the base member to provide a predetermined volume that compensates for a tissue volume loss when the implant is implanted on a bone structure.

14. The method of claim 13 further comprising, shaping the implant to be compliant with a bone structure.

15. The method of claim 13 wherein attaching the base member to the volume member includes attaching by one of (i) an adhesive; (ii) a fusion process; (iii) a bonding process; (iv) a heating method; (v) an electrochemical process; and (vi) a bonding agent that dissolves after the implant has been implanted.

16. The method of claim 13 wherein the base member is made from a metallic material and includes a plurality of perforations and at least one attachment member adapted to be secured to the bone structure by a bone screw.

17. The method of claim 13 wherein the volume member is greater in thickness than the base member.

18. The implant of claim 1 wherein the volume member is attached to the base member by one of (i) an adhesive; (ii) a heating method; (iii) an electrochemical process; and (iv) a bonding agent that dissolves into a human body.
